(19) United States
(12) Patent Application Publication
(10) Pub. No.: US 2016/0206179 A1
(43) Pub. Date: Jul. 21, 2016

(54) ASSISTIVE ROBOT ENDOSCOPIC SYSTEM WITH INTUITIVE MANEUVERABILITY FOR LAPAROSCOPIC SURGERY AND METHOD THEREOF

(71) Applicant: National Taiwan University, Taipei City (TW)

(72) Inventor: Ren C. LUO, Taipei City (TW)

(73) Assignee: NATIONAL TAIWAN UNIVERSITY, Taipei City (TW)

(21) Appl. No.: 14/597,672

(22) Filed: Jan. 15, 2015

(51) Int. Cl. A61B 1/00; A61B 19/00; A61B 1/04; A61B 1/313

(52) U.S. Cl. CPC A61B 1/00039; A61B 1/3132; A61B 19/2203; A61B 1/0016; A61B 1/04; A61B 2019/2211; A61B 2019/2269; Y10S 901/09

(57) ABSTRACT

Provided is an assistive robot endoscopic system, including a wireless gyroscope, measuring an intuitive motion of a user's (e.g., a surgeon) head, generating data based on the intuitive motion of the user's head and transmitting the data to a computer; a control system, receiving the data from the computer; and a laparoscope, having a robotic endoscope and automatically controlled by the control system based on the intuitive motion of the user's head. In addition, the present invention further provides an assistive robot endoscopic method.

### BACKGROUND OF THE INVENTION

1. Field of the Invention

The present invention relates to a robot, and more specifically to an assistive robot endoscopic system and method thereof.

2. The Prior Arts

A laparoscopy procedure or a minimally invasive surgery (MIS) is a unique technique for performing surgery. Over the last 10 years the use of this technique has expanded into intestinal surgery. In a laparoscopic surgery, several 0.5-1 cm incisions are created and may serve as the entry points into the abdomen. A tubular instrument known as a trochar is inserted at each incision. A laparoscope, a kind of specialized camera, is then passed through the trochar during the procedure. The laparoscope transmits images from the abdominal cavity to a high-resolution monitor in an operation room. This system may largely reduce the size of the incision without losing operation effectiveness. As such, the laparoscope may provide surgeons with an instant view while the instrument extends the reach of hands.

Some advanced endoscopes and instruments have been developed, such that a surgeon can perform interventions that cannot be performed by the conventional endoscopes. However, a number of assistants are still required to control an endoscopic device, and only a small working area is provided. This may lead to an unnatural cramped position from both surgeons and assistants. In addition, the assistants may not exactly follow the surgeons' instructions to move the endoscopic device to the right position.

Therefore, for the sake of meeting the requirement of providing a robotic system with low power consumption and high efficiency while performing surgery. As such, it is necessary to provide a robotic system and method thereof having high intuitivism, high safety, high stability and low cost.

### SUMMARY OF THE INVENTION

The primary objective of the present invention is to provide an assistive robot endoscopic system. The assistive robot endoscopic system may include a wireless gyroscope that measures an intuitive motion of a user's head, generates data based on the motion of the user's head and transmits the data to a computer; a control system that receives the data from the computer; and a laparoscope that has a robotic endoscope and is automatically controlled by the control system based on the intuitive motion of the user's head.

Preferably, the data generated by the wireless gyroscope may include rotation, orientation, angular velocity and angular acceleration information.

Preferably, the control system may include a driver and a PC-based programmable multi-axis controller (PMAC) motion control.

Preferably, the laparoscope may further include a plurality of servo motors, a shaft and a plurality of handles. The plurality of handles may be controlled by the plurality of servo motors.

Preferably, the data generated by the wireless gyroscope may be converted into position data through inverse kinematics.

Preferably, the assistive robot endoscopic system of the present invention may further include a foot pedal. The foot pedal may be configured as a switch for transferring the data to indicate the laparoscopic surgical operation status.

Preferably, the assistive robot endoscopic system of the present invention may further include a monitor. The monitor may display a real-time laparoscopic image taken by the robotic endoscope.

Preferably, according to a preferred embodiment of the present invention, a distance of the wireless transmission may be 20 m, but not limited to the present invention.

Moreover, the present invention further provides an assistive robot endoscopic method. The assistive robot endoscopic method may include the steps of measuring an intuitive motion of a user's head, generating data based on the motion of the user's head and transmitting the data to a computer by means of a wireless gyroscope; receiving the data from the computer by a control system; and automatically controlling a robotic endoscope of a laparoscope by the control system based on the intuitive motion of the user's head.

According to a preferred embodiment of the present invention, the assistive robot endoscopic method may further include a step of converting the data generated by the wireless gyroscope into position data through inverse kinematics.

According to a preferred embodiment of the present invention, the assistive robot endoscopic method may further include a step of transferring data to indicate the laparoscopic surgical operation status by a foot pedal. The foot pedal may be a switch.

According to a preferred embodiment of the present invention, the assistive robot endoscopic method may further include a step of displaying a real-time laparoscopic image taken by the robotic endoscope on a monitor.

### BRIEF DESCRIPTION OF THE DRAWINGS

The present invention can be understood in more detail by reading the subsequent detailed description in conjunction with the examples and references made to the accompanying drawings, wherein:

FIG. 1 is a schematic diagram showing an assistive robot endoscopic system according to a preferred embodiment of the present invention;

FIG. 2 is a schematic diagram illustrating a wireless gyroscope according to a preferred embodiment of the present invention;

FIG. 3 is a block diagram illustrating a control system and a laparoscope according to a preferred embodiment of the present invention;

FIG. 4 is a schematic diagram illustrating a robotic endoscope controlled by a wireless gyroscope according to the preferred embodiments of the present invention; and

FIG. 5 is a flowchart showing an assistive robot endoscopic method according to a preferred embodiment of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

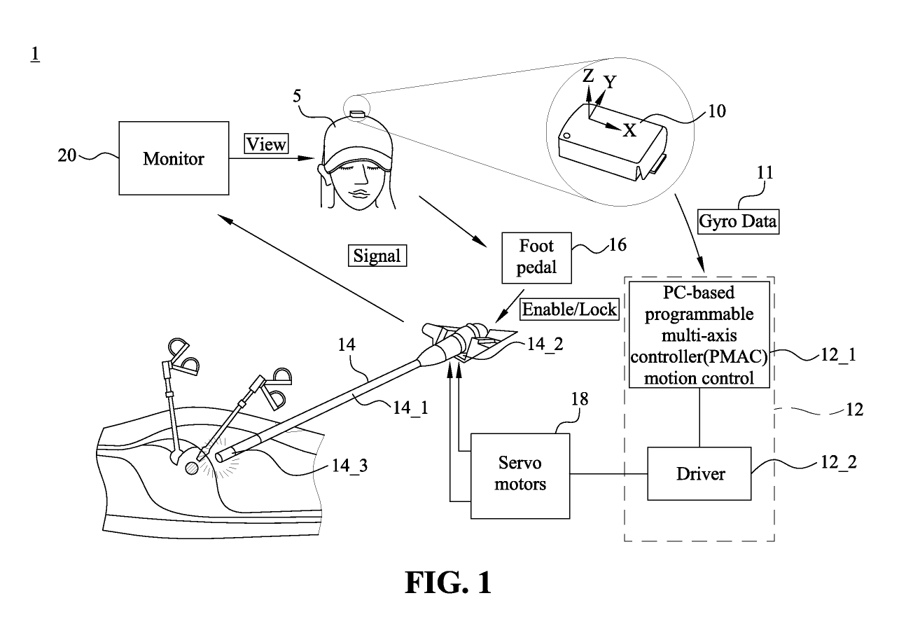

With regard to FIGS. 1-5, the drawings showing embodiments are semi-diagrammatic and not to scale and, particularly, some of the dimensions are for clarity of presentation and are shown exaggerated in the drawings. Similarly, although the views in the drawings for ease of description generally show similar orientations, this depiction in the drawings is arbitrary for the most part. Generally, the present invention can be operated in any orientation.

In light of the foregoing drawings, as shown in FIG. 1, the present invention provides an assistive robot endoscopic system 1. The assistive robot endoscopic system 1 includes a wireless gyroscope 10, a control system 12, a laparoscope 14, a foot pedal 16, a plurality of servo motors 18 and a monitor 20. The laparoscope 14 may include a shaft 14_1, a plurality of handles 14_2 and a robotic endoscope 14_3.

As shown in FIG. 1, a user or a surgeon 5 wears a wireless gyroscope 10 that measures his/her head movements. The measured data 11 may be processed by the control system 12, and may be used to actuate the laparoscope 14 while the foot pedal 16 is pressed. According, the surgeon 5 may see real-time laparoscopic images on a monitor 20 or on a head-mounted display (HMD) (not shown).

According to a preferred embodiment of the present invention, the laparoscope 14 may have two deflection degrees of freedom (DoF) and may turn to the field of view in humans

Moreover, a location-based algorithm may be developed to convert the measured data 11 generated by the wireless gyroscope into positions of laparoscope handles 14_2 through inverse kinematics. The plurality of servo motors 18 may be installed on the handles 14_2 of the laparoscope 14, such that the laparoscope 14 may be controlled by the wireless gyroscope 10 through the control system 12.

In addition, the laparoscope 14 of the present invention may be composed of a 10 mm articulating laparoscope equipped with the robotic endoscope 14_3, a fixed shaft 14_1 (about 40.6 cm long) and two handles 14_2. The robotic endoscope 14_3 may be a laparoscope camera. In other words, the laparoscope 14 may be regarded as a kind of mechanical arm with a camera. The camera may be placed at the end of the mechanical arm. Users 5 may adjust the direction of the camera to a target direction by controlling the plurality of handles 14_2 (e.g., two handles). According to an example of the present invention, the plurality of handles 14_2 may be controlled up and down, left and right. During an operation, the laparoscope 14 may be controlled a user through the wireless gyroscope 10 and the control system 12 of the present invention.

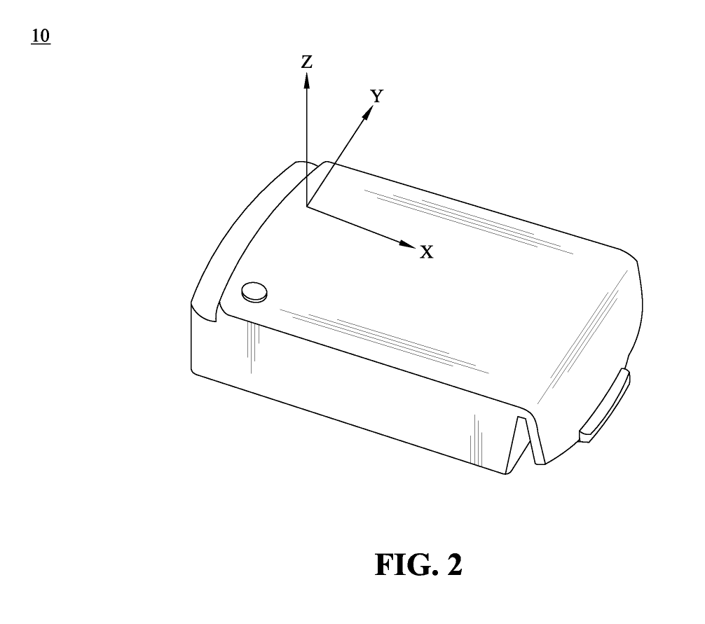

According to a preferred embodiment of the present invention, the wireless gyroscope 10 may be a device for measuring rotation, orientation, angular velocity and angular acceleration information based on the principle of angular momentum. As shown in FIG. 2, a wireless gyroscope 10 is illustrated. The amount of rotation, angular velocity and angular acceleration in three dimensions may be measured. The effective distance of wireless transmission may be 20 m long. Typically, the distance may be less than 2 m from a surgeon's head to a target position. The data 11 including rotation, orientation, angular velocity and angular acceleration information may also be sent to a PC by Bluetooth in accordance with a preferred example of the present invention.

In order to achieve a more precise control of the laparoscope 14, a relatively large gear ratio (e.g., 103:1) may be used in accordance with a preferred example of the present invention. Since the operation speed of the plurality of servo motors 18 is required to be relatively low, a driver having a smaller output current (e.g., 1A) may be used to control the plurality of servo motors 18.

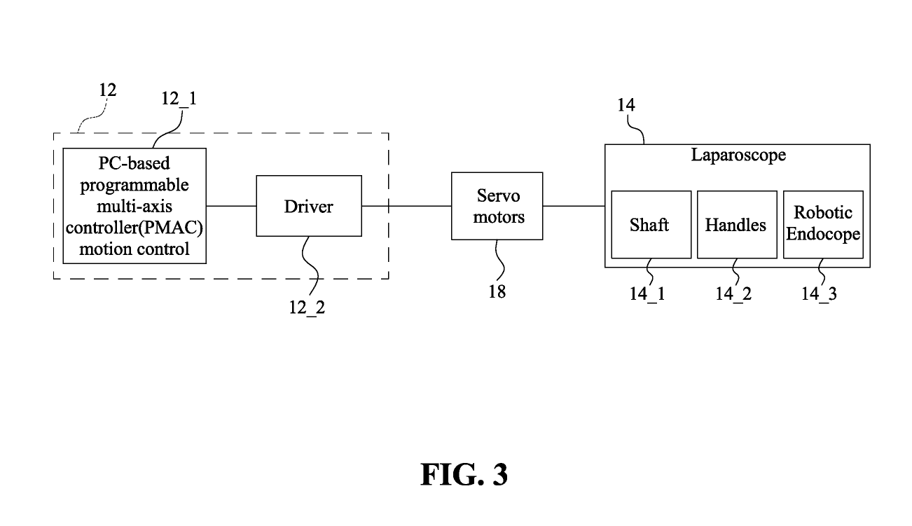

According to a preferred embodiment of the present invention, as shown in FIG. 3, a PC-based programmable multi-axis controller (PMAC) motion control 12_1 may be used to implement impedance and a velocity control algorithm. The PMAC 12 motion control may provide a servo interrupt time (e.g., 1 ms) for the control routine and may send a control command to the driver through a digital-to-analog (D/A) converter (not shown). As such, the driver 12_2 may be configured to a mode that may receive a torque command and may control a current control loop. That is to say, the robotic endoscope 14_3 of the laparoscope 14 and the plurality of servo motors 18 may be driven by the PMAC motion control 12_2 of the control system 12.

With regard to the control algorithm, the impedance control, the integral and the derivative control may be incorporated into the present invention. The impedance gain may be described as Equation (1).

$${Impedance}_{gain} ( s ) = \frac{{Q}_{u} {Θ}_{com} ( s )}{{following\_error}_{A} ( s )} \cdot \frac{1}{{A}_{tor\_vel}} \qquad ( 1 )$$

The adaptive Impedance_{gain}(s) may be used to compensate the velocity dropping due to the design of the constant impedance gain. The adaptive Impedance_{gain}(s) may depend on the changing of following error_{A}(s) and may be calculated by a suitable value to achieve the constant speed moving plan.

According to a preferred embodiment of the present invention, with the concept of the motion control, the wireless gyroscope 10 may be used to obtain signals from head rotary motions. With the developed programming algorithm, the position of the plurality of servo motors 18 moving the plurality of handles 14_2 may be determined. After initialization and all the setups, the output signal may be sent from the control system 12.

As for the control structure, the control system 12 with acceleration feed forward and gravity compensation may be applied. In other words, the control system 12 may approach the target position well and may have a low-stiffness response.

For the initialization, the starting direction of the wireless gyroscope 10 may be set as the origin, which is a reference point for the operation. In order to reduce noise, 100 gyroscope readings may be averaged when setting the origin. The averaged roll/pitch/yaw angle may be a new reference point.

Moreover, users/surgeons 5 may enable the laparoscope 14 to return to an original zero point in each operation. However, previous movements of the laparoscope may cause a deviation due to the backlash problem. According to a preferred embodiment of the present invention, a zero-point-correcting algorithm may be used to enable the laparoscope 14 to return to a zero point.

Besides, when the external force disturbs the laparoscope 14, the output torque may be increased to resist the force. Then, the laparoscope 14 may return to an equilibrium point with the output torque approaching to zero simultaneously. Therefore, the control strategy not only provides safety and compliance but also maintains the position precision.

A relationship between the load torque and the command torque is shown in Equation (2). Equation (2) may compute how much torque has to be generated for the system load. In other words, Equation (2) may determine how much torque is required to impose on the system 1 of the present invention.

$$[ {Θ}_{com} ( s ) - {Θ}_{feedback} ( s ) ] {K}_{Impedance} - \{ {ω}_{feedback} ( s ) [ {J}_{s} + ( C + \frac{{K}_{t} {K}_{b}}{R} ) ] + {τ}_{d} \} = {Torgue}_{com} \qquad ( 2 )$$

Θ_{com}(s): Command position
Θ_{feedback}(s): Actual feedback position
K_{impedance}: Impedance gain
J: Inertial of the system load
C: Viscosity coefficient of the system load
Kₜ: Torque constant
K_{b}: Back EMF coefficient
R: Resistance of the servo motor driver
τ_{d}: Disturbance torque

According to a preferred embodiment of the present invention, the assistive robot endoscopic system 1 may be assumed to have no gravitational torque because the end effector is relatively light.

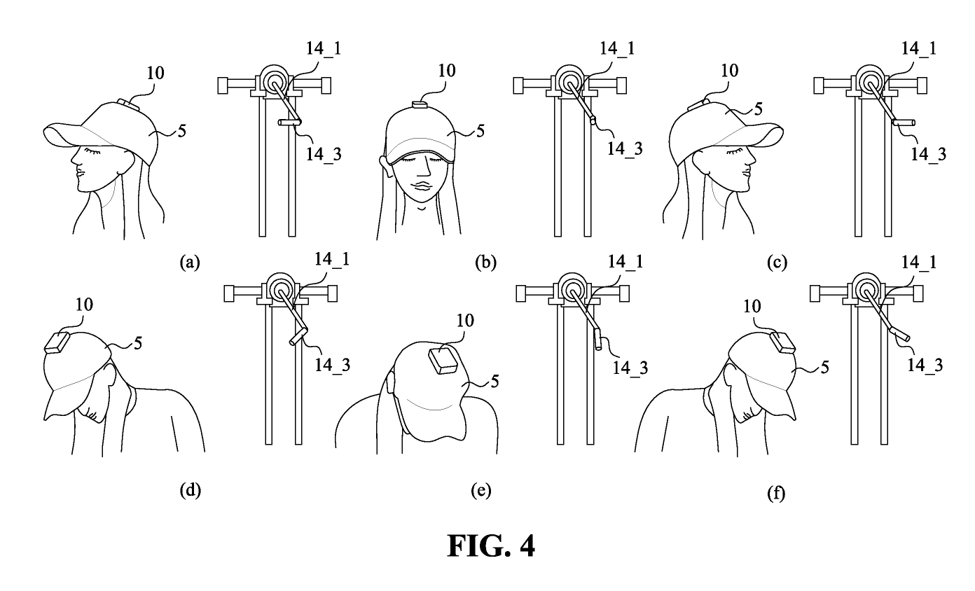

As shown in FIG. 4, the wireless gyroscope 10 may be worn on a user's head. The laparoscope 14 may be controlled by the wireless gyroscope 10. In other words, the robotic endoscope 14_3 of the laparoscope 14 may follow the trajectory of the user's head motion, as shown in FIG. 4(a)-(f).

Furthermore, the present invention provides an assistive robot endoscopic method. The assistive robot endoscopic method may include the following steps.

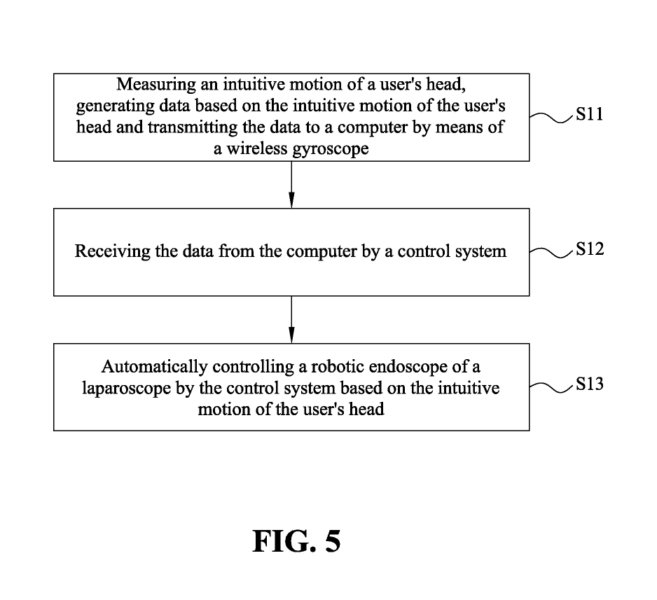

Referring to FIG. 5, the method begins with step S11 of measuring an intuitive motion of a user's head, generating data based on the intuitive motion of the user's head and transmitting the data to a computer by means of a wireless gyroscope.

Subsequently, at step S12, the data may be received from the computer by a control system. Then, proceed to step S13.

Then, in step S13, a robotic endoscope of a laparoscope may be automatically controlled by the control system based on the intuitive motion of the user's head.

According to a preferred embodiment of the present invention, the method may also include the steps of converting the data generated by the wireless gyroscope into position data through inverse kinematics; transferring data to indicate the laparoscopic surgical operation status by a foot pedal, wherein the foot pedal is a switch; and displaying a real-time laparoscopic image taken by the robotic endoscope on a monitor.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. An assistive robot endoscopic system, comprising:
a wireless gyroscope, measuring an intuitive motion of a user's head, generating data based on the intuitive motion of the user's head and transmitting the data to a computer;
a control system, receiving the data from the computer; and
a laparoscope, having a robotic endoscope and automatically controlled by the control system based on the intuitive motion of the user's head.

2. The assistive robot endoscopic system as claimed in claim 1, wherein the data generated by the wireless gyroscope comprises rotation, orientation, angular velocity and angular acceleration information.

3. The assistive robot endoscopic system as claimed in claim 1, wherein the control system comprises a driver and a PC-based programmable multi-axis controller (PMAC) motion control.

4. The assistive robot endoscopic system as claimed in claim 1, wherein the laparoscope further comprises a plurality of servo motors, a shaft and a plurality of handles, and the plurality of handles are controlled by the plurality of servo motors.

5. The assistive robot endoscopic system as claimed in claim 1, wherein the data generated by the wireless gyroscope is converted into position data through inverse kinematics.

6. The assistive robot endoscopic system as claimed in claim 1, further comprising a foot pedal, being a switch for transferring the data to indicate the laparoscopic surgical operation status.

7. The assistive robot endoscopic system as claimed in claim 1, further comprising a monitor, displaying a real-time laparoscopic image taken by the robotic endoscope.

8. The assistive robot endoscopic system as claimed in claim 1, wherein a distance of the wireless transmission is 20 m, and the wireless transmission comprises Bluetooth transmission.

9. An assistive robot endoscopic method, comprising the steps of:
measuring an intuitive motion of a user's head, generating data based on the intuitive motion of the user's head and transmitting the data to a computer by means of a wireless gyroscope;
receiving the data from the computer by a control system; and
automatically controlling a robotic endoscope of a laparoscope by the control system based on the intuitive motion of the user's head.

10. The assistive robot endoscopic method as claimed in claim 9, wherein the data generated by the wireless gyroscope comprises rotation, orientation, angular velocity and angular acceleration information.

11. The assistive robot endoscopic method as claimed in claim 9, wherein the control system comprises a driver and a PC-based programmable multi-axis controller (PMAC) motion control.

12. The assistive robot endoscopic method as claimed in claim 9, wherein the laparoscope further comprises a plurality of servo motors, a shaft and a plurality of handles, and the plurality of handles are controlled by the plurality of servo motors.

13. The assistive robot endoscopic method as claimed in claim 9, further comprising a step of converting the data generated by the wireless gyroscope into position data through inverse kinematics.

14. The assistive robot endoscopic method as claimed in claim 9, further comprising a step of transferring data to indicate the laparoscopic surgical operation status by a foot pedal, wherein the foot pedal is a switch.

15. The assistive robot endoscopic method as claimed in claim 9, further comprising a step of displaying a real-time laparoscopic image taken by the robotic endoscope on a monitor.

16. The assistive robot endoscopic method as claimed in claim 9, wherein a distance of the wireless transmission is 20 m, and the wireless transmission comprises Bluetooth transmission.
